(19) United States
(12) Patent Application Publication
(10) Pub. No.: US 2025/0238575 A1
(43) Pub. Date: Jul. 24, 2025

(54) METHOD FOR IDENTIFYING THE KEY MOLECULAR REGULATION PATH DETERMINING A PARTICULAR CELL

(71) Applicant: KOREA ADVANCED INSTITUTE OF SCIENCE AND TECHNOLOGY, Daejeon (KR)

(72) Inventors: Kwang-Hyun CHO, Daejeon (KR); Nam Hee KIM, Daejeon (KR)

(21) Appl. No.: 18/981,928

(22) Filed: Dec. 16, 2024

(30) Foreign Application Priority Data
Jan. 23, 2024 (KR) 10-2024-0010444

(51) Int. Cl. G06F 30/20

(52) U.S. Cl. CPC G06F 30/20

(57) ABSTRACT

A method of identifying a cell state regulation path is described, including the steps of setting a state of each node constituting an expanded network generated from a Boolean network corresponding to a biomolecular network of a particular cell to a value of a predetermined given initial state of the Boolean network; selecting a test subject node from diff-FBLs having complementary states in the initial state of the Boolean network and in a given target state, among the Boolean network FBLs, and perturbing the selected test subject node by computer simulation; testing whether the perturbation changes the values of all nodes in the diff-FBLs from initial state values to target state values; and, if the test subject node passes the test, determining that the test subject node is a master regulator, which must be controlled to transition the Boolean network from the initial state to the target state.

### CROSS-REFERENCE TO RELATED APPLICATION

The priority under 35 USC § 119 of Korean Patent Application 10-2024-0010444 filed Jan. 23, 2024 is hereby claimed, and the disclosure thereof is hereby incorporated herein by reference, in its entirety, for all purposes.

### TECHNICAL FIELD

The present invention relates to a technique for determining a key molecule that determines a cell phenotype and a signaling path of key molecules using a computer simulation.

### RELATED ART

Cells are characterized by their robustness to external signals and noise, and their ability to maintain their identity. However, since the successful reprogramming of cells through OSKM (Oct3/4, Sox2, Klf4 and c-Myc), it has been known that it is possible to change cell fate through specific perturbations in different contexts, such as the differentiation-inducing treatment of leukemia with ATRA (all-trans retinoic acid) treatment and the differentiation induction through YAP (yes-associated protein).

From these experimental observations, it has been known that not all molecules have the same response, but only certain conditions or specific molecular regulation cause such drastic changes. However, among numerous molecules that make up a cell, it is very difficult to identify which ones play a key role in changing the cell's fate.

Furthermore, even if the target molecule, the molecule that plays a key role in changing cell fate, is acknowledged, how regulating the expression of the target molecule changes cell fate remains an open question.

### PRIOR ART REFERENCE

### Patent Reference

U.S. Pat. No. 10,799,474 (2020.10.13.)—Method of treating leukemia utilizing somatic cell reprogramming
US 2023-0364064 (2023.11.16)—Use of tetrandrine in combination with all-trans retinoic acid in preparation of medicament for treating pneumoconiosis
KR 2023-0146054 (2023.10.18)—Small Molecule Activators of YAP Transcriptional Activity for Regenerative Organ Repair
KR 2452629 B1 (2022.10.04.)—A control method for driving any state of a Boolean network to a boundary state of the basin of a desired attractor by using a minimum temporary perturbation
KR 2196064 B1 (2020.12.22.)—Method for estimating a signal flow in a complex signal transfer network using only the structure of the network and device for the same
KR 1483789 B1 (2015.01.12.)—Method for analyzing network characteristic and computer-readable medium and apparatus for the same

### DETAILED DESCRIPTION OF THE INVENTION

### Technical Problem

The determination of the two cellular states of interest is closely related to a positive feedback, among motifs constituting a complex network. The positive feedback is characterized as bistable switches and plays an important role in the transition between stable states. This fact has been recognized in many studies, and the present invention is based on this fact and pays particular attention to the pathway of positive feedback in molecular networks.

In addition, the present invention utilizes a Boolean network model that can express cell fate using simpler parameters to define a central structure determined by logical connections as well as network structural information.

A Boolean network is a nonlinear modeling method in which the state of a node is represented by ‘1’ or ‘0’, and the state space can be tracked through the updates of the regulatory relations expressed by Boolean formula. Within this state space, states that are no longer updated and can remain stable are defined as ‘attractors’ or ‘attractor states’, and the set of convergence spaces defined by the attractors and the states that converge to each of these attractors can be defined as an ‘attractor landscape’.

The present invention focuses on various control methodologies to stabilize the state of the cell and utilizes a novel algorithm developed using two of the following methodologies.

The first methodology is the LDOI (Logical Domain of Influence) method. It is a technique that extends the network into an expanded network by corresponding the logic information of the network to each node, and then searches for a set of nodes whose state can be fixed with a single update regardless of the initial state when the state of a specific node selected from the expanded network is fixed. This method is useful for finding target nodes that ensure the convergence of the network to a particular target state. However, it has the limitation that it assumes the entire state space of the network to search for target nodes, which inevitably leads to unnecessary targets in the process of controlling the transition between two states of interest among the states that the network can have.

The second methodology is the DEPC (Differentially Expressed Positive Circuits) method. It is a method of finding top nodes by selecting feedback structures in which the values of two attractors differ which are the two states of interest, among the states of the network, and it has a very low complexity, but it has the limitation of low accuracy.

The present invention provides a novel methodology utilizing the two methodologies described above, which can present a target that can alter cell fate, along with the structure which is critical when the target is controlled.

### Technical Solution

The present invention may provide a method of identifying a cell state regulation path, comprising the steps,
setting, by a computing device, a state of each node constituting an expanded network generated from a Boolean network corresponding to a biomolecular network of a particular cell to a value of a predetermined given initial state of the Boolean network;
selecting, by the computing device, a test subject node, which is one of the nodes belonging to diff-FBLs, defined as FBLs having complementary states in the initial state of the Boolean network and in a given target state, among FBLs belonging to the Boolean network, and perturbs the selected test subject node by computer simulation;
testing, by the computing device, whether perturbing the test subject node changes the values of all nodes in the diff-FBLs from values in the initial state to values in the target state; and
if the test subject node passes the test, determining, by the computing device, that the test subject node is a master regulator, which must be controlled to transition the Boolean network from the initial state to the target state.

The method of identifying a cell state regulation path further comprises the steps, determining, by the computing device, whether any of the diff-FBLs in the Boolean network are not directly connected to other diff-FBLs;
if the diff-FBLs which are not directly connected to other diff-FBLs exist, determining, by the computing device, a set of nodes that serve as bridges connecting the diff-FBLs to each other among the remaining nodes whose state values do not change between the initial state and the target state in the Boolean network; and
defining, by the computing device, a substructure consisting of the diff-FBLs and the set of FBLs which act as bridges as a canalizing kernel of the Boolean network.

The Boolean network comprises a first set of nodes corresponding to N molecules expressed in the particular cell and a first set of links connecting the first set of nodes to each other, wherein the first set of links comprises a first type of link and a second type of link, wherein the first type of link is a link wherein activation of a source node connected to a starting point of the link has a positive effect on activation of a target node connected to a destination point of the link, and the second type of link is a link wherein activation of a source node connected to a starting point of the link has a negative effect on activation of a target node connected to a destination point of the link, and wherein the expanded network may be an equivalent network to the Boolean network. Further, the expanded network may comprise: a second set of nodes corresponding to the first set of nodes, a third set of nodes defined as having complementary values with respect to the second set of nodes, a fourth set of combinatorial nodes executing a logical AND operation of two nodes included in the expanded network, and a second set of links connecting the second set of nodes, the third set of nodes, and the fourth set of combinatorial nodes to each other. The second set of links may comprise only the first type of links.

The testing step may comprise: a first testing step determining a state value of the nodes in the expanded network having a determined state according to predetermined first and second rules; and a second testing step determining a state value of the remaining nodes in the expanded network whose state is not determined by the first testing step. The first rule comprises a rule for determining an active state of a second node connected to a link starting from a first node determined to be active among the nodes of the expanded network, and the second rule comprises a rule for determining an inactive state of a node defined as having a value complementary to a node determined to be active among the nodes of the expanded network, wherein both the first node and the second node may be nodes whose state is determined according to an expression state of a single molecule of the particular cell among the nodes of the expanded network, respectively.

The second testing step may be executed only after the test subject node has passed the first testing step. If the nodes corresponding to any of the nodes included in the diff-FBLs among the nodes of the expanded network whose state values have been determined as a result of executing the first testing step have values where the Boolean network has the target state, the test subject node may be determined to have passed the first test.

In accordance with another aspect of the present invention, a computing device comprising a non-volatile storage device and a processing part may be provided. The processing part is configured to execute a method of identifying a cell state regulation path by reading and executing a program recorded on the non-volatile storage device. The program may comprise the instructions for causing the processing part to execute the steps of setting a state of each node constituting an expanded network generated from a Boolean network corresponding to a biomolecular network of a particular cell to a value of a predetermined given initial state of the Boolean network; selecting a test subject node, which is one of the nodes belonging to diff-FBLs, defined as FBLs having complementary states in the initial state of the Boolean network and in a given target state, among FBLs belonging to the Boolean network, and perturbing the selected test subject node by computer simulation; testing whether perturbing the test subject node changes the values of all nodes in the diff-FBLs from values in the initial state to values in the target state; and, if the test subject node passes the test, determining that the test subject node is a master regulator, which must be controlled to transition the Boolean network from the initial state to the target state.

The program may further comprise instructions for causing the processing part to further execute the steps of: determining whether any of the diff-FBLs in the Boolean network are not directly connected to other diff-FBLs; if the diff-FBLs which are not directly connected to other diff-FBLs exist, determining a set of FBLs that serve as bridges between the diff-FBLs among the remaining FBLs in the Boolean network that are not directly connected to other diff-FBLs; and defining a substructure consisting of the diff-FBLs and the set of FBLs which act as bridges as a canalizing kernel of the Boolean network.

The Boolean network comprises a first set of nodes corresponding to N molecules expressed in the particular cell and a first set of links connecting the first set of nodes to each other, wherein the first set of links comprises a first type of link and a second type of link, wherein the first type of link is a link wherein activation of a source node connected to a starting point of the link has a positive effect on activation of a target node connected to a destination point of the link, and the second type of link is a link wherein activation of a source node connected to a starting point of the link has a negative effect on activation of a target node connected to a destination point of the link, and wherein the expanded network may be an equivalent network to the Boolean network. The expansion network may comprise: a second set of nodes corresponding to the first set of nodes, a third set of nodes defined as having complementary values with respect to the second set of nodes, a fourth set of combinatorial nodes executing a logical AND operation of two nodes included in the expanded network, and a second set of links connecting the second set of nodes, the third set of nodes, and the fourth set of combinatorial nodes to each other. The second set of links may comprise only the first type of links.

In accordance with another aspect of the present invention, there may be provided a non-volatile storage device on which a program readable by a computing device is recorded. The program may comprise the instructions causing the computing device to execute the steps of setting a state of each node constituting an expanded network generated from a Boolean network corresponding to a biomolecular network of a particular cell to a value of a predetermined given initial state of the Boolean network; selecting a test subject node, which is one of the nodes belonging to diff-FBLs, defined as FBLs having complementary states in the initial state of the Boolean network and in a given target state, among FBLs belonging to the Boolean network, and perturbing the selected test subject node by computer simulation; testing whether perturbing the test subject node changes the values of all nodes in the diff-FBLs from values in the initial state to values in the target state; and, if the test subject node passes the test, determining that the test subject node is a master regulator, which must be controlled to transition the Boolean network from the initial state to the target state.

The program may further comprise instructions for causing the computing device to further execute the steps of: determining whether any of the diff-FBLs in the Boolean network are not directly connected to other diff-FBLs; if the diff-FBLs which are not directly connected to other diff-FBLs exist, determining a set of nodes that serve as bridges connecting the diff-FBLs to each other among the remaining nodes whose state values do not change between the initial state and the target state in the Boolean network; and defining a substructure consisting of the diff-FBLs and the set of FBLs which act as bridges as a canalizing kernel of the Boolean network.

The Boolean network comprises a first set of nodes corresponding to N molecules expressed in the particular cell and a first set of links connecting the first set of nodes to each other, wherein the first set of links comprises a first type of link and a second type of link, wherein the first type of link is a link wherein activation of a source node connected to a starting point of the link has a positive effect on activation of a target node connected to a destination point of the link, and the second type of link is a link wherein activation of a source node connected to a starting point of the link has a negative effect on activation of a target node connected to a destination point of the link, and wherein the expanded network may be an equivalent network to the Boolean network. The expanded network may comprise: a second set of nodes corresponding to the first set of nodes, a third set of nodes defined as having complementary values with respect to the second set of nodes, a fourth set of combinatorial nodes executing a logical AND operation of two nodes included in the expansion network, and a second set of links connecting the second set of nodes, the third set of nodes, and the fourth set of combinatorial nodes to each other. The second set of links may comprise only the first type of links.

### Advantageous Effects

According to the present invention, key molecules that determine the phenotype of a particular cell can be identified using computer simulations, and key molecular regulation paths consisting of the key molecules can be.

### BRIEF DESCRIPTION OF DRAWING

FIG. 1 illustrates a concept and method of network extension for use in one example of the present invention.

FIGS. 2a to 2d are diagrams to illustrate how to define a master regulatory DEPC that constitutes a Boolean network, according to one example of the present invention.

FIG. 3 is a simple Boolean network exemplified to illustrate the process of finding a master regulator provided in accordance with one example of the present invention.

FIG. 4a shows the initial state (first attractor) of the Boolean network of FIG. 3, and FIG. 4b shows the target state (second attractor) of the Boolean network of FIG. 3.

FIG. 5a shows a total of five FBLs found in the Boolean network of FIG. 3.

FIG. 5b shows the diff-FBLs observed among the five FBLs presented in FIG. 5a.

FIG. 5c shows the state values of the diff-FBLs in the initial and target states of the Boolean network.

FIG. 6 is an expanded network of the Boolean network presented in FIG. 3, which was expanded using the method presented in FIG. 1.

FIG. 7 depicts a situation in which the expanded network (72) of FIG. 6 has the initial state shown in FIG. 4a.

FIGS. 8a and 8b are intended to illustrate a first test among tests that track nodes whose state is conclusively determined when the state of node (x02) is perturbed to determine whether node (x02) is a master regulator.

The FIG. 9 shows the clustering of nodes whose values cannot be determined even by the methods presented in FIGS. 8a and 8b.

FIG. 10 separately shows only the first and second clusters presented in FIG. 9.

FIG. 11 is a flowchart illustrating a method for determining a master regulator corresponding to a key protein that determines a particular cell phenotype, according to one example of the present invention.

FIG. 12 is a flowchart illustrating how to define the core structure of a given Boolean network, the so-called canalizing kernel, according to one example of the present invention.

FIG. 13 is a flowchart illustrating how to define the core structure of a given Boolean network, the so-called canalizing kernel, according to one example of the present invention.

FIG. 14 shows a canalizing kernel and identified targets involved in the transition from EpiSC to ESC defined by applying the method of the present invention to a stem cell reprogramming network (Yachie-Kinoshita et al.). The right panel is the canalizing kernel and the hierarchically determined sequence defined by the method of the present invention when Klf4 is upregulated.

FIG. 15 shows a canalizing kernel and target involved in the trans-differentiation from B cells to macrophage defined by applying the method of the present invention to a trans-differentiation network (Collombet et al.). The right panel is the hierarchically determined sequence in each factors of canalizing kernel after Cebpa overexpression.

### BEST MODE

Examples of the present invention will be described hereinafter with reference to the accompanying drawings. However, the present invention is not limited to the examples described herein and may be implemented in many other forms. The terminology used herein is intended to aid in understanding the examples and is not intended to limit the scope of the invention. In addition, singular expressions used herein include plural expressions unless the context clearly indicates the contrary.

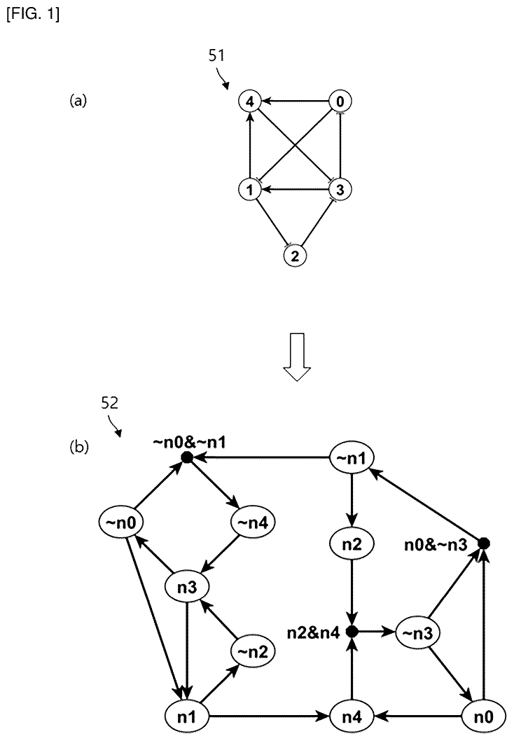

FIG. 1 illustrates a concept and method of network extension for use in one example of the present invention.

In (a) of FIG. 1, an example of a Boolean network (51) is shown. The structural information of the Boolean network (51) consists of five nodes represented by integers of ‘0’ to ‘4’ and links connecting two nodes to each other. In general, the structural information of a Boolean network may consist of information defining the nodes and links constituting it. Each node has a value indicating whether the biomolecule (e.g., gene or protein) corresponding to each node is expressed or not, which can be either ‘1’ or ‘0’. A value of ‘1’ for a node indicates that the biomolecule corresponding to the node is expressed, and a value of ‘0’ for a node indicates that the biomolecule corresponding to the node is not expressed. Each link has a unidirectional direction: links represented by arrow heads are first type of links, where the activation of the source node positively affects the activation of the target node, and links represented by bare heads are second type of links, where the activation of the source node negatively affects the activation of the target node. As such, the Boolean network may include both types of links. The equations defining the state of the Boolean network (51) are f₀=NOT σ₃, f₁=(NOT σ₀) OR σ₃, f₂=NOT σ₁, f₃=(NOT σ₂) OR (NOT σ₄), f₄=σ₀ OR σ₁.

In (b) of FIG. 1, an expanded network (52) is shown that is an extension of the Boolean network (51) shown in (a) of FIG. 1.

In this specification, the expanded network (52) is an equivalent network to the Boolean network (51). The expanded network (52) also includes a plurality of nodes and links. The links of the expanded network (52), unlike the Boolean network (51), comprise only the first type of links.

In order to make the links of the expanded network (52) comprise only the first type of links, the expanded network (52) comprises a first set of nodes corresponding to the nodes constituting the Boolean network (51) and a second set of nodes having values complementary to the values of the nodes of the first set. In other words, a single node labeled with the reference symbol “0” in the Boolean network (51) shown in (a) of FIG. 1 is presented as expanded with a node labeled with the reference symbol “no” and a complementary node labeled with the reference symbol “˜n0” in the expanded network (52) shown in (b) of FIG. 1.

The expanded network (52) further comprises combinatorial operation nodes, which are nodes for representing cases where a combination of two or more nodes affects one other node. For example, the combination of nodes (n2) and (n4) by the logical operator AND affects node (˜n3), thus representing the combinatorial operation node (n2&n4) between nodes (n2), (n4), and (˜n3). As another example, node (n3) is influenced by node (˜n4) and node (˜n2), but only by node (˜n4) and node (˜n2) independently, and not by the AND combination of node (˜n4) and node (˜n2). Therefore, no separate combinatorial operation node is shown between nodes (n3), (˜n4), and (˜n2). In (b) of FIG. 1, each combinatorial operation node is represented by a black dot.

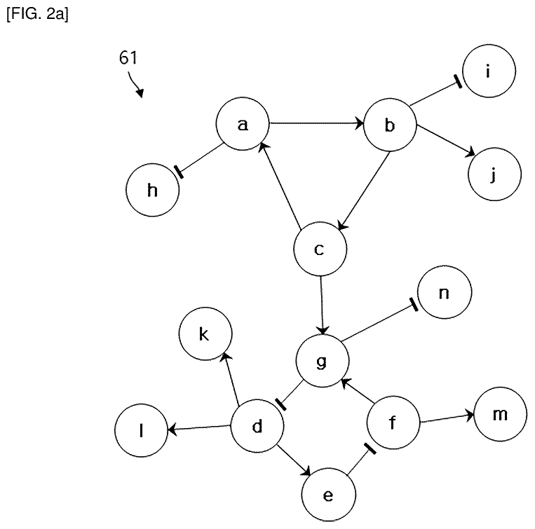

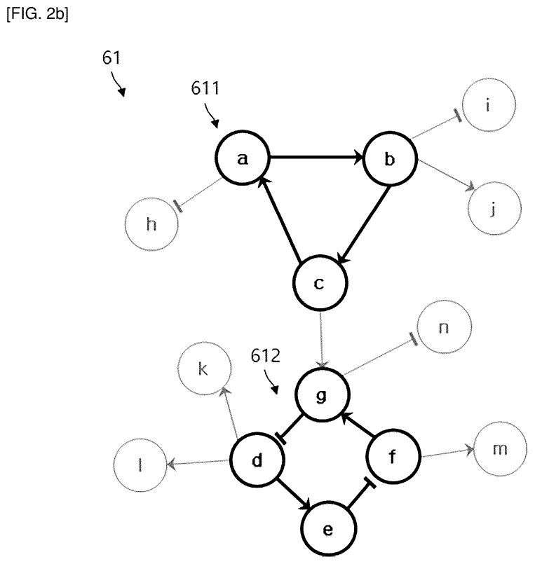

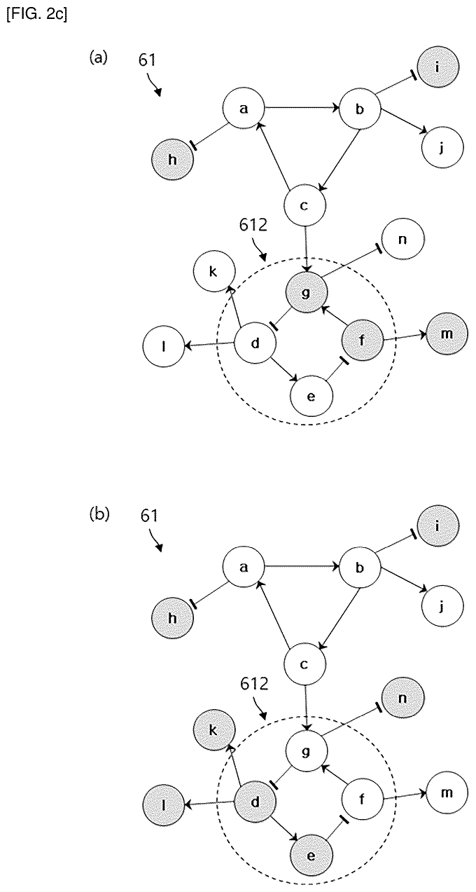

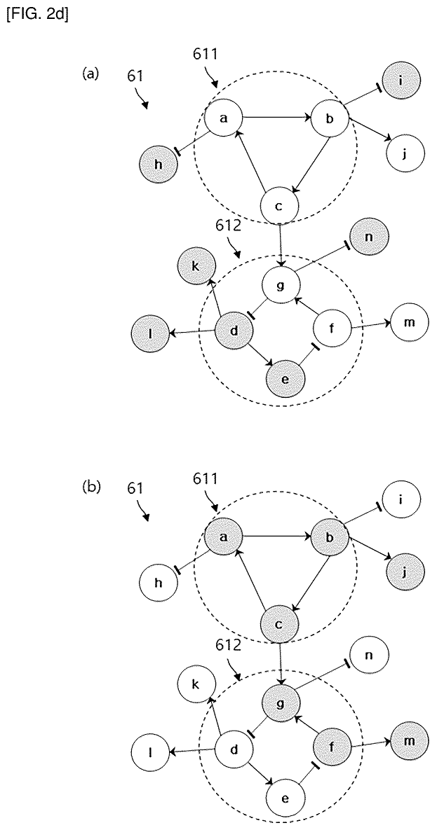

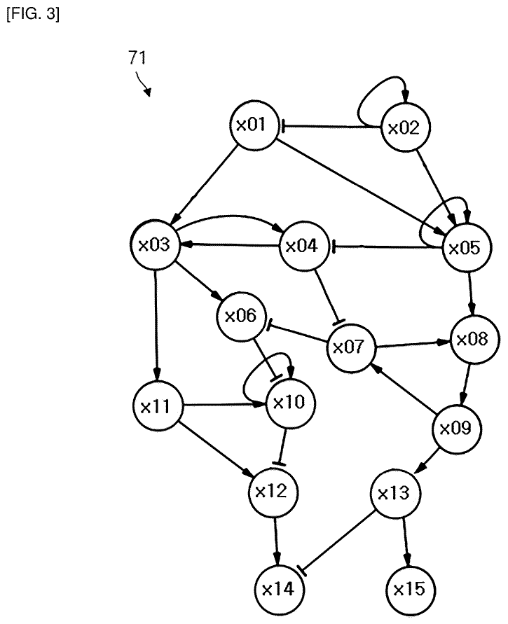

FIGS. 2a to 2d are diagrams to illustrate how to define a master regulatory DEPC that constitutes a Boolean network, according to one example of the present invention.

FIG. 2a illustrates a Boolean network (61) comprising 14 nodes, labeled “a” to “n” by reference, and links connecting them. The Boolean network (61) may comprise one or more loops partially having a positive feedback (hereinafter, simply “FBL”). The Boolean network (61) illustrated in FIG. 2a includes a first FBL (611) comprising node a, node b, and node c, and a second FBL (612) comprising node d, node e, node f, and node g.

A computing device provided in accordance with one example of the present invention may be configured to obtain and process structural information data regarding structural information of the Boolean network. The computing device may be configured to identify FBLs constituting the Boolean network from the structural information data. It may be understood that each FBL is a partially existing sub-Boolean network.

FIG. 2b is a bolded redrawing of the first and second FBLs observed in the Boolean network of FIG. 2a.

FIG. 2C depicts two states, a first attractor and a second attractor, selected from a number of states that the Boolean network (61) may have.

In general, each node in a Boolean network can only have one of two values: ‘0’ or ‘1’, so the total number of states that a Boolean network with N nodes can have is 2\hat{ }N. Among these states, there can also be one or more attractors that are in a state where the state no longer changes over time. The state, attractor, and basin of Boolean network are described in Korean Patent No. KR 1483789 B1 (Jan. 12, 2015).

The two states represented in FIG. 2c can both be different attractors.

In FIG. 2C, a node represented in white color is a node having a first value of a binary value, and a node represented in dark color is a node having a second value of a binary value. The first and second values are ‘1’ and ‘0’, respectively, or ‘0’ and ‘1’, respectively.

FIG. 2c (a) illustrates a first attractor of the Boolean network (61), and FIG. 2c (b) illustrates a second attractor of the Boolean network (61). As described above, the Boolean network (61) includes two FBLs (611, 612), but only the second FBL (612) is the FBL that has complementary states in the first attractor and the second attractor.

Here, when a given FBL has complementary states in the first attractor and the second attractor, it indicates the state values of each and every node constituting the given FBL have complementary states in the first attractor and the second attractor. If the state values of any one node constituting a given FBL are not complementary in the first and second attractors (i.e., they have the same state value), it cannot be said that the given FBL has complementary states in the first and second attractors.

That is, node d, node e, node f, and node g constituting the second FBL (612) in the first attractor have a value of 2, a value of 2, a value of 1, and a value of 1, respectively, of the binary values, and node d, node e, node f, and node g constituting the second FBL (612) in the second attractor have a value of 1, a value of 1, a value of 1, a value of 2, and a value of 2, respectively, of the binary values, and thus have complementary values to each other.

In contrast, node a, node b, and node c constituting the first FBL (611) in the first attractor have a value of 1, a value of 1, and a value of 1, respectively, of the binary values, and node a, node b, and node c constituting the second FBL (612) in the second attractor also have a value of 1, a value of 1, and a value of 1, respectively, of the binary values, and thus do not have complementary values to each other.

In the present invention, the part of a given Boolean network that constitutes a feedback loop can be defined as FBL. For example, it can be assumed that there is a first FBL in the Boolean network. Then, the first attractor and the second attractor can be selected among the states of the Boolean network. If the first FBL has complementary states in the first attractor and the second attractor, then the first FBL can be defined as belonging to a category named diff-FBL. In other words, if an FBL in a given Boolean network has complementary states in two different attractors of the Boolean network, the FBL is defined as belonging to a category named diff-FBL.

Referring back to FIG. 2c, the Boolean network (61) has two FBLs (611, 612), but it has only one diff-FBL 612 for the combination of the first attractor and the second attractor.

FIG. 2d depicts two selected states, the second attractor and the third attractor, out of several states that the Boolean network (61) can have.
(a) in FIG. 2d illustrates a second attractor of the Boolean network (61), and (b) in FIG. 2d illustrates a third attractor of the Boolean network (61).

The second attractor of the Boolean network (61) shown in (a) of FIG. 2d is the same as the second attractor of the Boolean network (61) shown in (b) of FIG. 2c.

In the second attractor and the third attractor, the first FBL (611) has a complementary state to each other. Furthermore, the second FBL (612) in the second attractor and the third attractor are complementary to each other. Thus, both the first FBL (611) and the second FBL (612) are the diff-FBLs.

Thus, in the combination of the second attractor and the third attractor, the Boolean network (61) has two diff-FBLs.

As described above, according to the present invention, a given Boolean network having a determined structure has a determined fixed number of FBLs. In this case, the Boolean network may have a number of diff-FBLs less than the fixed number of FBLs. Further, the diff-FBLs may be defined, by selecting two different attractors of the Boolean network, to be dependent on the two selected attractors. Of the two attractors described above, one of the attractors may refer to an initial state as addressed in the present invention, and the other state may refer to a target state.

One example of the present invention aims to find a master regulator, which is a key node that needs to be regulated in order for the state of the Boolean network to make a state transition from the initial state to the target state. The master regulator may be referred to herein as a target node. If the master regulator is properly identified, a drug acting on a biomolecule corresponding to the master regulator (target node) can be selected as a target drug.

In one example, the initial state may be a state in which the cell is a cancer cell. The target state may be a state in which the cell is dead, a state in which the cell is no longer differentiating, or a state in which the cell is a normal cell. In other words, the present invention may be used to identify biomolecules that are key to manipulating to cause a state transition of a cell, called a cancer cell, to another desirable state. Once the biomolecule is identified, one or more drugs or combinations of drugs from a class of drugs that are effective in modulating the expression of the biomolecule can be selected as target drugs.

FIG. 3 is a simple Boolean network (71) exemplified to illustrate the process of finding a master regulator provided in accordance with one example of the present invention.

The master regulator may be defined in a situation where the diff-FBLs defined by the pair of initial state (first attractor) and target state (second attractor) of a predetermined Boolean network are defined. The master regulator may be any one of the nodes included in the Boolean network. In the initial state, the state of the master regulator (target node) has an undesired state. Upon changing the state of the master regulator of the Boolean network having the initial state to another state, the states of all the diff-FBLs are transitioned from the values in the initial state to the values in the target state.

In a Boolean network having the initial state, if any one node is selected to change its state (=undesired initial state) to another state (=desired target state), and the values of all the diff-FBLs of the initial state are changed to the values of all the diff-FBLs of the target state, the selected node may be considered a master regulator.

The master regulator can exist among all nodes in all diff-FBLs. There may be two or more master regulators among all nodes in all diff-FBLs.

A master moderator verification method provided in accordance with one example of the present invention may verify that any one selected node included in all the diff-FBLs is qualified as a master moderator. If there are a total of Nd nodes included in all the diff-FBLs, the master moderator verification method may be executed independently for each of the Nd nodes included in all the diff-FBLs, i.e., the master moderator verification method may be executed Nd times.

The Boolean network (71) illustrated in FIG. 3 is shown to include a total of 15 nodes, labeled x01 to x15, and includes the first type of links and the second type of links.

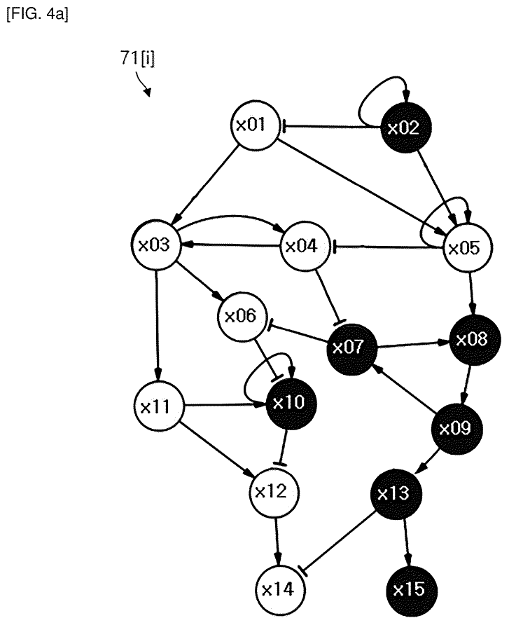

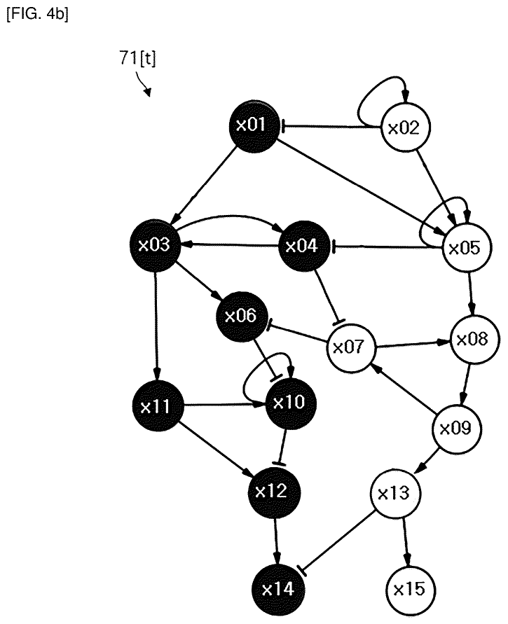

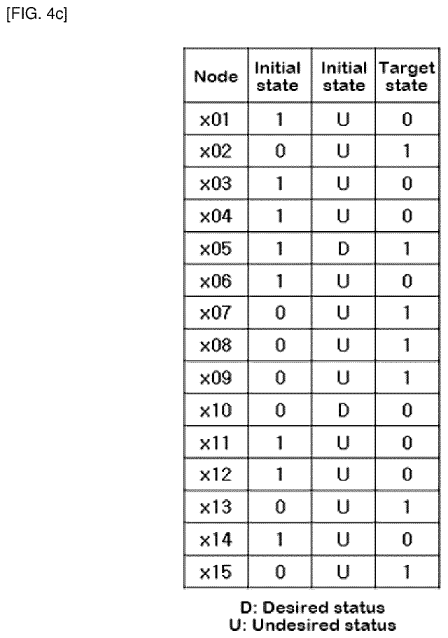

FIG. 4a illustrates an initial state (first attractor) of the Boolean network (71) of FIG. 3, and FIG. 4b illustrates a target state (second attractor) of the Boolean network (71) of FIG. 3.

For example, if utilized in a treatment process for a cancer patient of the present invention, the initial state may be a cancer cell state of the cell represented by the Boolean network (71), and the target state may be a normal or dead state of the cell.

In contrast, if the present invention is utilized for the purpose of studying the mutual transition process between cancer cells and normal cells, the initial state may be a normal cell state or a dead state of the cell represented by the Boolean network (71), and the initial state may be a cancer cell state of the cell.

Alternatively, for example, the initial state may be a disease state other than a cancerous state of the cell represented by the Boolean network (71), and the target state may be a normal or dead state of the cell.

As such, the status of the initial state and the target state may be selected by a person utilizing the present invention.

In FIG. 4a, the Boolean network (71) having an initial state is represented by reference numeral ‘71i’, and in FIG. 4b, the Boolean network (71) having a target state is represented by reference numeral ‘71t’.

In FIGS. 4a and 4b, each node is represented as a circle, wherein the black node has a first state value and the white node has a second state value. The first state value and the second state value may be ‘0’ and ‘1’, respectively. Alternatively, the first state value and the second state value may be ‘1’ and ‘0’, respectively. Hereinafter, for ease of explanation, the first state value and the second state value are assumed to be ‘0’ (=white node) and ‘1’ (=black node), respectively.

In the initial state of FIG. 4a and the target state of FIG. 4b, the two nodes whose values are the same are node (x05) and node (x10), while the other nodes have different values in the initial state and the target state. Therefore, in the initial state, node (x05) and node (x10) already have the desired values, while the other nodes have undesired values.

FIG. 4c shows an example of the values of each node in the initial state of FIG. 4a and the target state of FIG. 4b.

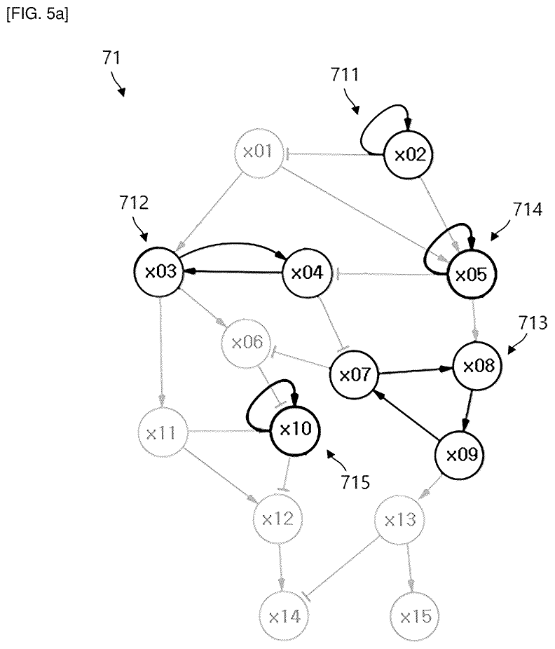

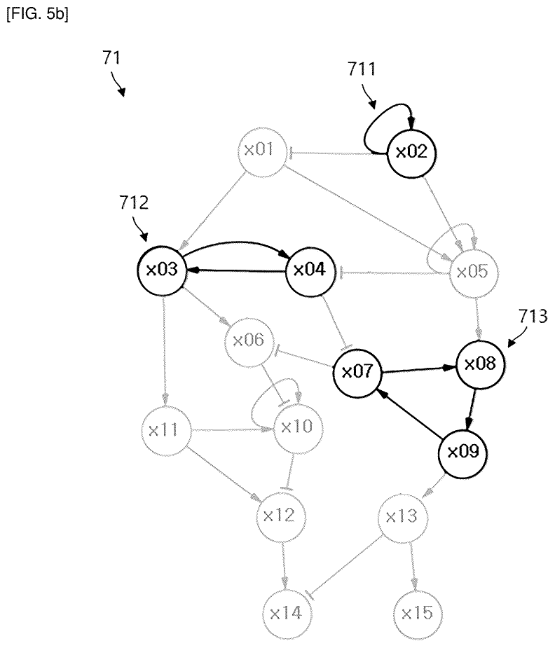

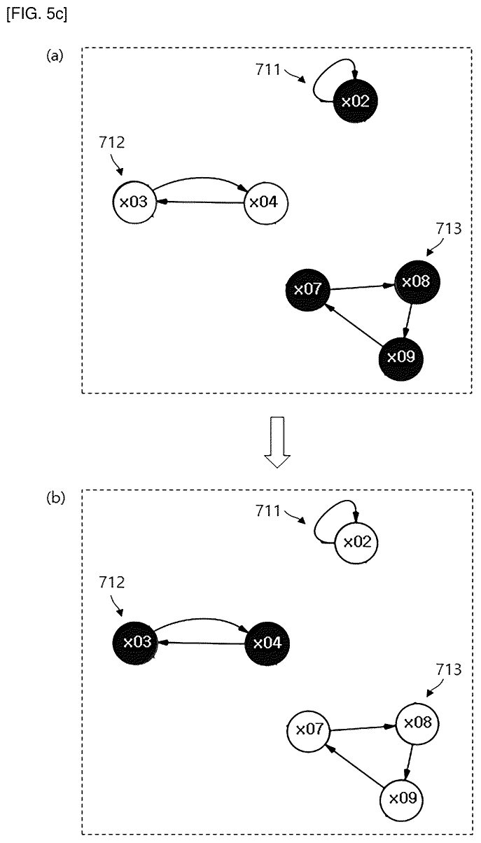

FIG. 5a illustrates a total of five FBLs found in the Boolean network (71) of FIG. 3.

The first FBL (711) consists of node (x02), the second FBL (712) consists of node (x03), and node (x04), the third FBL (713) consists of node (x07), node (x08), and node (x09), the fourth FBL (714) consists of node (x05), and the fifth FBL (715) consists of node (x10).

FIG. 5b shows the diff-FBLs observed among the five FBLs presented in FIG. 5a.

Referring to FIG. 4c, the state values in the initial state and the state values in the target state of the nodes constituting the first FBL (711) are all different from each other, the state values in the initial state and the state values in the target state of the nodes constituting the second FBL (712) are all different from each other, and the state values in the initial state and the state values in the target state of the nodes constituting the third FBL (713) are all different from each other. Thus, the first FBL (711), the second FBL (712), and the third FBL (713) are all diff-FBLs.

However, referring to FIG. 4c, the state values in the initial state and the state values in the target state of the nodes constituting the fourth FBL (714) are identical to each other, and the state values in the initial state and the state values in the target state of the nodes constituting the fifth FBL (715) are identical to each other. Therefore, the fourth FBL (714) and the fifth FBL (715) are not diff-FBLs.

FIG. 5c shows the state values of the diff-FBLs in the initial and target states of the Boolean network (71).

In (a) of FIG. 5c, the state values of the first FBL (711), the second FBL (712), and the third FBL (713) are shown when the Boolean network (71) has the above initial state.

In (b) of FIG. 5c, the state values of the first FBL (711), the second FBL (712), and the third FBL (713) are shown when the Boolean network (71) has the above target state.

The first FBL (711) has {x02}={0} in the initial state and {x02}={1} in the target state.

The second FBL (712) has {x03, x04}={1, 1} in the initial state and {x03, x04}={0, 0} in the target state.

The third FBL (713) has {x07, x08, x09}={0, 0, 0} in the initial state and {x07, x08, x09}={1, 1, 1} in the goal state.

As described above, the present invention seeks to identify a master regulator, a node that plays a key role in the process of controlling the Boolean network (71) such that the diff-FBLs defined with respect to a predetermined initial state and a target state selected for the Boolean network (71) have values in the target state.

The master regulator may be expected to be present among the nodes included in the diff-FBLs defined with respect to a predetermined initial state and target state selected for the Boolean network (71). Accordingly, a master regulator verification method may be performed on each of the nodes included in the diff-FBLs to determine whether each of the nodes included in the diff-FBLs is a master regulator.

In the example of FIG. 5c, it can be verified whether node (x0), node (x3), node (x4), node (x7), node (x8), node (x9) are master regulators, respectively.

In one example of the present invention, the master regulator verification method is not executed for the remaining other nodes (x1 to x02, x5 to x6, and x10 to x15) that constitute the Boolean network (71).

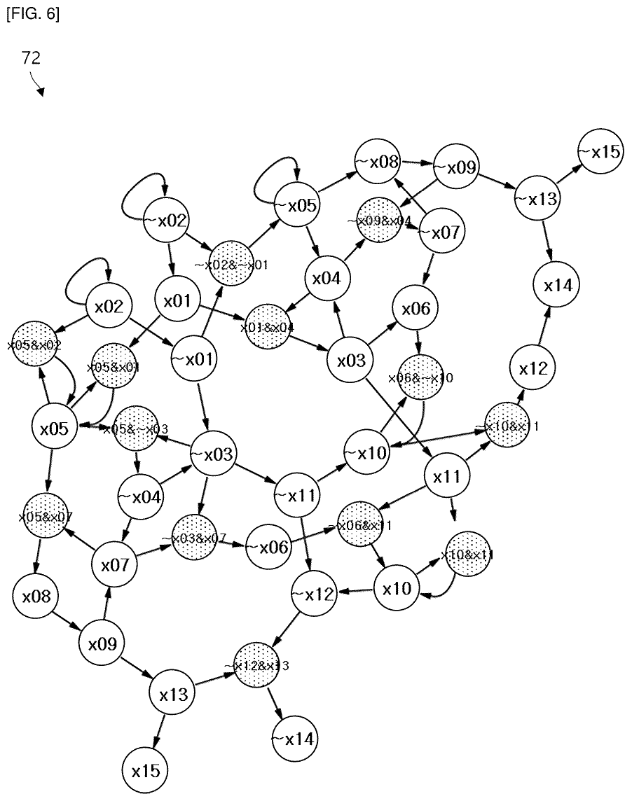

FIG. 6 is an expanded network (72) that expands the Boolean network (71) presented in FIG. 3 using the method presented in FIG. 1.

The links included in the expanded network (72) are all the first type of links.

The expanded network (72) includes a first set of nodes corresponding to the nodes comprising the Boolean network (71), and a second set of nodes having values complementary to the values of the first set of nodes. For example, node (x02) of the Boolean network (71) shown in FIG. 3 is provided as expanded to node (x02) and node (˜x02) in the expanded network (72) shown in FIG. 6.

Hereinafter, for ease of explanation, the “combinatorial operation node” described in FIG. 1 is represented as a circle filled with dots in FIG. 6.

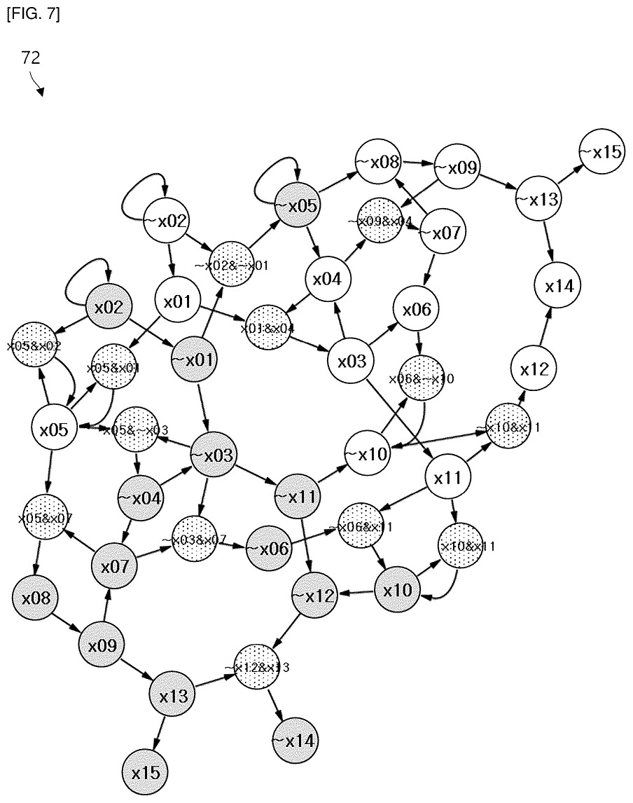

FIG. 7 depicts a situation in which the expanded network (72) of FIG. 6 has the initial state shown in FIG. 4a.

As shown in FIG. 4c, node (x01), node (x03), node (x04), node (x05), node (x06), node (x11), node (x12), and node (x14) have a value of ‘1’ in the initial state, so these nodes are represented by ‘white’ circles in FIG. 7. Conversely, node (˜x01), node (˜x03), node (˜x04), node (˜x05), node (˜x06), node (˜x11), node (˜x12), and node (˜x14) have complementary values and are represented by gray circles in FIG. 7.

Also, as shown in FIG. 4c, node (x02), node (x07), node (x08), node (x09), node (x10), node (x13), and node (x15) have a value of ‘0’ in the initial state, so these nodes are represented by ‘gray’ circles in FIG. 7. Conversely, node (˜x02), node (˜x07), node (˜x08), node (˜x09), node (˜x10), node (˜x13), and node (˜x15), which have complementary values, are represented by ‘white’ circles in FIG. 7.

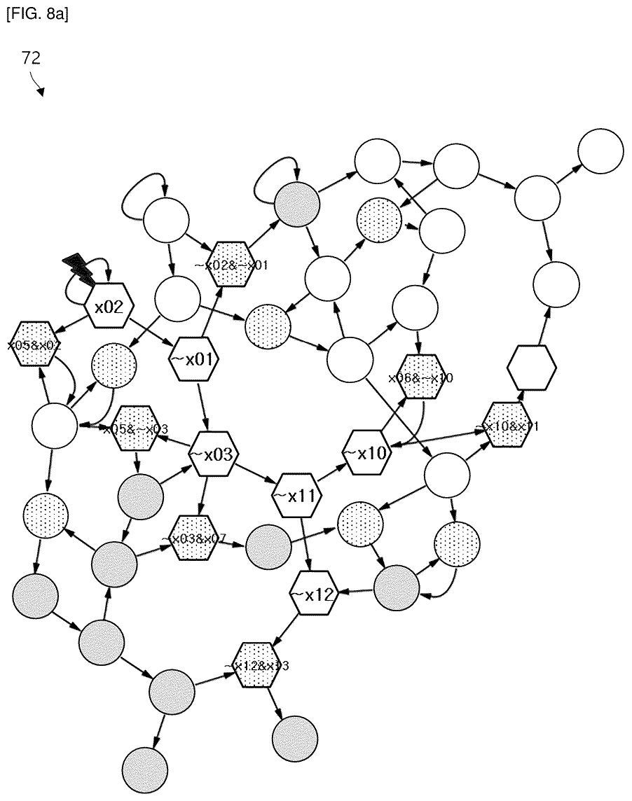

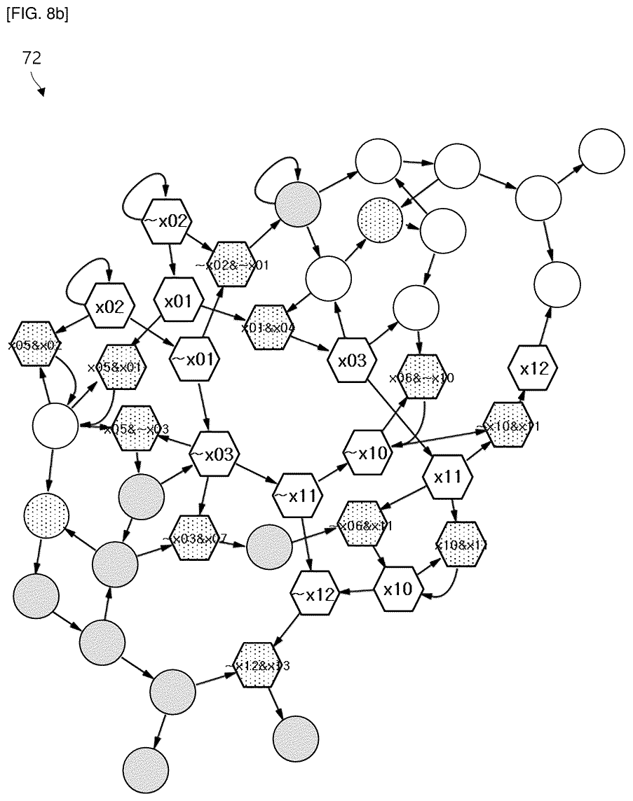

Now, whether a master regulator exists among the nodes belonging to the first FBL (711), the second FBL (712), and the third FBL (713) described above can be determined by executing a predetermined test method for each of the nodes. Hereinafter, FIGS. 8a, 8b, 9, and 10 present an example of a test method for testing whether node (x02) is a master regulator. This example may be generalized to constitute one example of the present invention.

FIGS. 8a and 8b are intended to illustrate a first test among tests that tracks nodes whose state is conclusively determined when the state of node (x02) is perturbed to determine whether node (x02) is a master regulator.

The first test includes the following rules. To illustrate these rules, a distinction is made between combinatorial nodes, which constitute the expanded network (72), and other nodes, which are molecular nodes. The combinatorial nodes are nodes that include the ‘&’ operator. Further, the molecular nodes, other than the combinatorial nodes, are represented by the reference symbols x01 to x15, and ˜x01 to ˜x15.

The molecular node may be considered to be a node in the expanded network generated from the Boolean network modeling a particular cell, the state of which is determined by the expression state of a single molecule of the particular cell.

Each arrow shown in FIG. 6, FIG. 7, FIG. 8a, and FIG. 8b represents a link, wherein a node connected to the starting point of the link may be referred to as a source node and a node connected to the destination point of the link may be referred to as a target node.

First, other molecular nodes (target nodes) connected to the molecular node having the determined active state also have the determined active state. For example, in FIG. 8a, if the molecular node (x02) has a determined value and its determined state is active, the molecular node (˜x01) directly connected by the link originating from the molecular node (x02) also has a determined state of active. In other words, if the molecular node that is the source is determined to be active, the molecular node that is the target connected to the molecular node that is the source by the link is also determined to be active. Even if there are two or more source molecular nodes (source nodes) connected to a target molecular node, when even one of the source molecular nodes is determined to be active, the target molecular node is also determined to be active.

Even if any molecule node has a determined state, if that state is inactive, the any molecule node does not determine the state of any target molecule node connected to the any molecule node.

Second, a combinatorial operation node connected to a molecular node with a determined active state loses the meaning of its existence. For example, in FIG. 8a, the combinatorial operation node (x05&x02) connected to node (x02) is only determined by x02 because the value of x05 is determined, i.e., the function of the ‘&’ operator in the combinatorial operation node (x05&x02) disappears.

Third, a molecular node defined as having a complementary state to a molecular node with a determined active state is determined to have an inactive state. For example, since molecular node (x02) is determined to be active, molecular node (˜x02), which has a complementary relationship to it, is determined to be inactive. As another example, if molecular node (˜x10) is determined to be active, then molecular node (x10) having a complementary relationship to it is determined to be inactive.

When the state of the molecular node (x02) is perturbed to determine whether the molecular node (x02) is a master regulator, the state of the molecular node (x02) is definitively determined. The node whose state is definitively determined by the perturbation is represented by a hexagon in FIG. 8a.

Hereafter, the molecular node may be referred to simply as a node.

Hereinafter, the description will be provided with reference to FIG. 8a.

Referring to FIG. 8a, the process of tracing the propagation path of an active state, based on structural information of the expanded network (72), when perturbing the test subject node (x02), to determine whether it is a master regulator, can be illustrated.

Hereafter, ‘1’ means an active state and ‘0’ means an inactive state.

Before the perturbation, node (x02) has a value of ‘0’, so after the perturbation, node (x02) has a value of ‘1’.

Since the state of node (x02) is confirmed to be ‘1’, the state of the target node (˜x01) connected to it is confirmed to be ‘1’.

Since the state of node (x02) has been determined to be ‘1’, the ‘&’ operator of the combinatorial operation node (x05&x02) connected to it loses its meaning, and as a result, the combinatorial operation node (x05&x02) can be represented as a hexagon.

Since the state of node (˜x01) has been determined to be ‘1’, the ‘&’ operator of the combinatorial operation node (˜x02&˜x01) connected to it loses its meaning, and as a result, the combinatorial operation node (˜x02&˜x01) can be represented as a hexagon.

Since the state of node (˜x01) is confirmed to be ‘1’, the state of the target node (˜x03) connected to it is confirmed to be ‘1’.

Since the state of node (˜x03) has been determined to be ‘1’, the ‘&’ operator in the combinatorial operation node (x05&˜x03) connected to it loses its meaning, and as a result, the combinatorial operation node (x05&˜x03) can be represented as a hexagon.

Since the state of node (˜x03) has been determined to be ‘1’, the ‘&’ operator on the combinatorial operation node (˜x03&x07) connected to it loses its meaning, and as a result, the combinatorial operation node (˜x03&x07) can be represented as a hexagon.

Since the state of node (˜x03) is confirmed to be ‘1’, the state of the target node (˜x11) connected to it is confirmed to be ‘1’.

Since the state of node (˜x11) is resolved to ‘1’, the state of the target node (˜x10) connected to it is resolved to ‘1’.

Since the state of node (˜x11) is resolved to ‘1’, the state of the target node (˜x12) connected to it is resolved to ‘1’.

Since the state of node (˜x12) has been determined to be ‘1’, the ‘&’ operator on the combinatorial operation node (˜x12&x13) connected to it loses its meaning, and as a result, the combinatorial operation node (˜x12&x13) can be represented as a hexagon.

Since the state of node (˜x10) has been determined to be ‘1’, the ‘&’ operator of the combinatorial operation node (x06&˜x10) connected to it loses its meaning, and as a result, the combinatorial operation node (x06&˜x10) can be represented as a hexagon.

Since the state of node (˜x10) has been determined to be ‘1’, the ‘&’ operator on the combinatorial operation node (˜x10&x11) connected to it loses its meaning, and as a result, the combinatorial operation node (˜x10&x11) can be represented as a hexagon.

Hereinafter, the description will be provided with reference to FIG. 8b.

Referring to FIG. 8b, a process for determining values of nodes that are complementary to nodes having a determined active state may be described, according to the process described with reference to FIG. 8a.

All nodes with a state value determined by the process shown in FIG. 8a have an active state (‘1’), while all nodes with a state value determined by the process shown in FIG. 8b have an inactive state (‘0’).

Since the state of node (x02) is determined to be ‘1’, the state of its complementary node (˜x02) is determined to be ‘0’.

Since the state of node (˜x02) has been determined, the ‘&’ operator in the combinatorial operation node (˜x02&˜x01) connected to it loses its meaning, and as a result, the combinatorial operation node (˜x02&˜x01) can be represented as a hexagon. Since the state of node (˜x01) is resolved to ‘1’, the state of its complementary node (x01) is resolved to ‘0’.

Since the state of node (x01) has been determined, the ‘&’ operator in the combinatorial operation node (x05&x01) connected to it loses its meaning, and as a result, the combinatorial operation node (x05&x01) can be represented as a hexagon.

Since the state of node (x01) has been determined, the ‘&’ operator on the combinatorial operation node (x01&x04) connected to it loses its meaning, and as a result, the combinatorial operation node (x01&x04) can be represented as a hexagon.

Since the state of node (˜x03) is resolved to ‘1’, the state of its complementary node (x03) is resolved to ‘0’.

Since the state of node (˜x11) is resolved to ‘1’, the state of its complementary node (x11) is resolved to ‘0’.

Since the state of node (˜x11) has been determined, the ‘&’ operator in the combinatorial operation node (˜x06&˜x11) connected to it loses its meaning, and as a result, combinatorial operation node (˜x06&˜x11) can be represented as a hexagon.

Since the state of node (˜x11) has been determined, the ‘&’ operator on the combinatorial operation node (x10&˜x11) connected to it loses its meaning, and as a result, the combinatorial operation node (x10&˜x11) can be represented as a hexagon.

Since the state of node (˜x12) is resolved to ‘1’, the state of its complementary node (x12) is resolved to ‘0’.

As can be seen in FIGS. 8a and 8b, the perturbation to node (x02) causes its state value to be determined to be ‘1’. Thus, the state of the first FBL (711) when the Boolean network (71) has the target state is achieved, as shown in (b) of FIG. 5c. From this perspective, the node (x02) fulfills one of the requirements of being a master regulator.

Furthermore, as can be seen in FIGS. 8a and 8b, the state value of node (˜x03) is determined to be ‘0’ because its state value is determined to be ‘1’ by the perturbation to node (x02).

If the state value of the node (x04) can be determined to be ‘0’, the state of the second FBL (712) when the Boolean network (71) has the above target state can be achieved, as shown in (b) of FIG. 5c. However, the state value of the node (x04) cannot be determined by the above first test presented in FIGS. 8a and 8b.

Now, if the state of the second FBL (712) and the state of the third FBL (713) when the Boolean network (71) has the above target state are achieved, then the node (x02) fulfills all of the above requirements for being a master regulator. Therefore, it is necessary to observe the state of the node (x04), which is a node included in the second FBL (712), and the node (x07), node (x08), and node (x09), which are nodes included in the third FBL (713). However, the states of node (x04), node (x07), node (x08), and node (x09) are not included in the nodes whose states have been definitively determined according to the node state determination rules presented in FIGS. 8a and 8b.

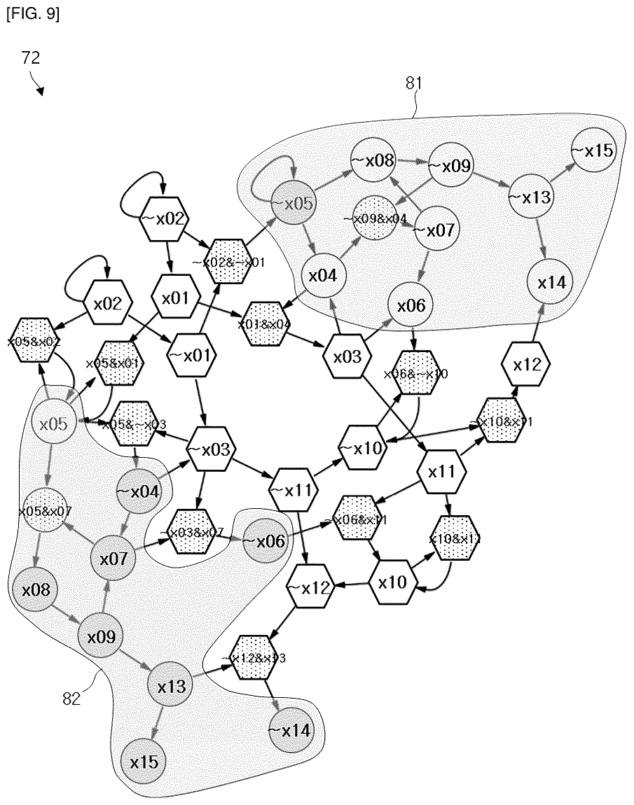

Therefore, in addition to the node state determination rules presented in FIGS. 8a and 8b, it is necessary to determine the states of node (x04), node (x07), node (x08), and node (x09) by the method described later with reference to FIGS. 9 and 10.

FIGS. 9 and 10 are intended to illustrate a second test among tests that tracks nodes whose state is conclusively determined when the state of node (x02) is perturbed to determine whether node (x02) is a master regulator.

The second test for node (x02) may be executed after node (x02) has passed the first test. The test subject node (x02) is determined to have passed the first test if, as a result of the first test for the node (x02), the nodes included in the diff-FBLs among the nodes whose state values have been determined have values where the Boolean network (71) has the target state.

Among the nodes whose state values are determined as a result of the first test, the nodes included in the diff-FBLs (711 to 713) are node (x02) and node (x03). Since the determined state values of the node (x02) and the node (x03) have values of ‘1’ and ‘0’, respectively, which are values where the Boolean network (71) has the target state, the test subject node (x02) may be determined to have passed the first test.

FIG. 9 shows the clustering of nodes whose values cannot be determined by the methods presented in FIGS. 8a and 8b.

The nodes whose states are not determined are categorized into first cluster (81) and second cluster (82).

Each of the first cluster (81) and the second cluster (82) is determined by selecting the test subject node as node (x02). If the test subject node is selected as a node other than node (x02), the number and composition of the clusters defined after the first test may be different.

FIG. 10 separately shows only the first cluster (81) and second cluster (82) presented in FIG. 9.

In the first cluster (81), nodes (˜x07), (˜x08), and (˜x09) associated with the third FBL (713) form a loop, and in the second cluster (82), nodes (x07), (x08), and (x09) associated with the third FBL (713) form a loop.

At this time, to satisfy the state of the second FBL (712) and the state of the third FBL (713) when the Boolean network (71) has the above target state, a second test, wherein the node (x04) of the second cluster (82) is given a state value of ‘0’, all of node (x07), node (x08), and node (x09) are given a status value of ‘1’, node (˜x04) of the first cluster (81) is given a status value of ‘1’, and all of nodes (˜x07), (˜x08), and (˜x09) are given a status of ‘0’, can be performed.

When the second test is executed, if no logical contradiction occurs in both the first cluster (81) and the second cluster (82), the node (x04) can be determined to have a state value of ‘0’ and the node (x07), the node (x08), and the node (x09) to have a state value of ‘1’. Thus, as shown in (b) of FIG. 5c, the state of the second FBL (712) and the state of the third FBL (713) where the Boolean network (71) has the target state are achieved. From this perspective, the node x02 fulfills all of the above requirements as a master regulator. As a result, the node (x02) is determined to be a master regulator.

In contrast, when the second test is executed, if a logical contradiction occurs in either the first cluster (81) or the second cluster (82), it cannot be determined that node (x04) has a state value of ‘0’ and node (x07), node (x08), and node (x09) all have a state value of ‘1’. Therefore, as shown in (b) of FIG. 5c, the state of the second FBL (712) and the state of the third FBL (713) where the Boolean network (71) has the target state are not achieved. From this perspective, the node (x02) does not fulfill all of the above requirements for being a master regulator. As a result, the node (x02) is determined not to be a master regulator.

In the example shown in FIG. 10, when running the second test, node (x02) is determined to be the master regulator because there is no logical contradiction in both the first cluster (81) and the second cluster (82).

The first and second tests shown in FIGS. 8a, 8b, 9, and 10 may be executed individually/repeatedly for each of the nodes included in the diff-FBLs. It may be determined that zero, one, or two or more of the tested nodes are master regulators as described above.

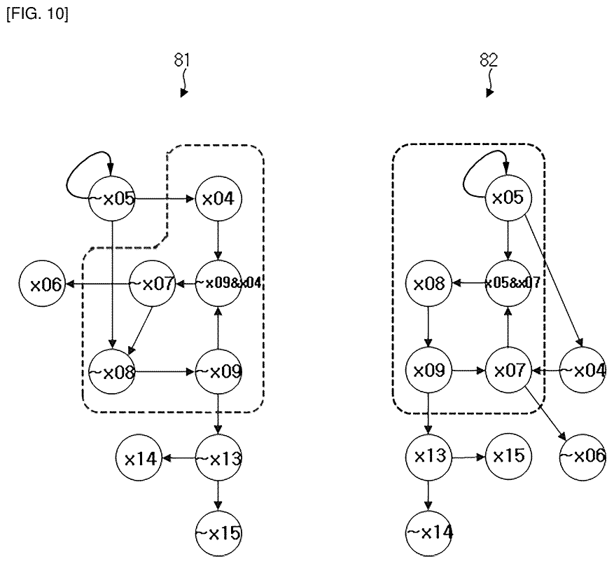

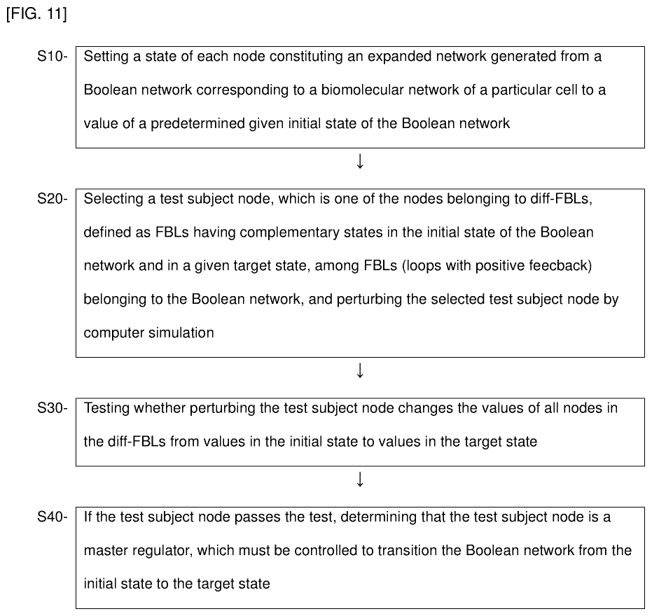

FIG. 11 is a flowchart illustrating a method for determining a master regulator corresponding to a key protein that determines a particular cell phenotype, according to one example of the present invention.

In step (S10), the computing device may set a state of each node constituting the expanded network obtained from the Boolean network corresponding to the biomolecular network of the particular cell to a value of a given initial state of the Boolean network.

Step (S10) may include the following steps (S11) to (S14).

In step (S11), the computing device may obtain from a predetermined storage device information defining a Boolean network corresponding to a biomolecular network of a particular cell.

In step (S12), the computing device may generate an expanded network from the Boolean network, and temporarily or semi-permanently store the information defining the expanded network in a predetermined storage device.

In step (S13), the computing device may determine or obtain a value of each node of the Boolean network in a predetermined initial state of the Boolean network, and determine or obtain a value of each node of the Boolean network in a predetermined target state of the Boolean network. The process of the computing device obtaining the values of the nodes in the initial and target states of the Boolean network may comprise reading the corresponding values of the nodes from a predetermined storage device.

In step (S14), the computing device may set a state value of each node constituting the expanded network to a value corresponding to the initial state of the Boolean network.

For example, the Boolean network is the network (71) of FIG. 3, and the expanded network is the network (72) of FIG. 6, wherein a given initial state of the Boolean network is the initial state shown in FIG. 4c, and an example wherein the state of each node constituting the expanded network is set to a value of the initial state is shown in FIG. 7.

In step (S20), the computing device may select, among the FBLs (loops with positive feedback) belonging to the Boolean network, one node (test subject node) belonging to diff-FBLs, which are FBLs having complementary states in the initial state of the Boolean network and in a given target state, to perturb by computer simulation.

Step (S20) may include the following steps (S21) to (S24).

In step (S21), the computing device may determine the FBLs belonging to the Boolean network.

In step (S22), the computing device may determine the values of the nodes in the FBLs where the Boolean network is in the initial state, and determine the values of the nodes in the FBLs where the Boolean network is in the target state.

In step (S23), the computing device may determine, among the FBLs, FBLs whose values in the initial state and values in the target state are complementary to each other, and determine the determined FBLs as diff-FBLs.

In step (S24), the computing device may select one of the nodes belonging to the diff-FBLs and run a simulation of perturbing the selected node in the expanded network to determine whether the selected node is a master regulator. Perturbing the selected node may cause the initial state value of the selected node to be changed to a complementary value.

For example, the FBLs (loops with positive feedback) are the loops (711-715) shown in FIG. 5a, the target state is the target state shown in FIG. 4c, the diff-FBLs are the loops (711-713) shown in FIG. 5c, and an example of the one test subject node is the node (x02) shown in FIG. 8a.

In step (S30), the computing device may test whether, upon perturbing the test subject nodes, the values of all nodes in the diff-FBLs change from values in the initial state to values in the target state.

The testing may include the first test, described with reference to FIGS. 8a and 8b, and the second test, described with reference to FIGS. 9 and 10. The second test for the test subject node may be executed sequentially after the test subject node passes the first test.

In step (S40), the computing device may determine that, if the test subject node passes the test, the test subject node is a master regulator for transitioning the Boolean network from the initial state to the target state.

In other words, perturbing the test subject node can determine that the state of the Boolean network transitions from the initial state to the target state.

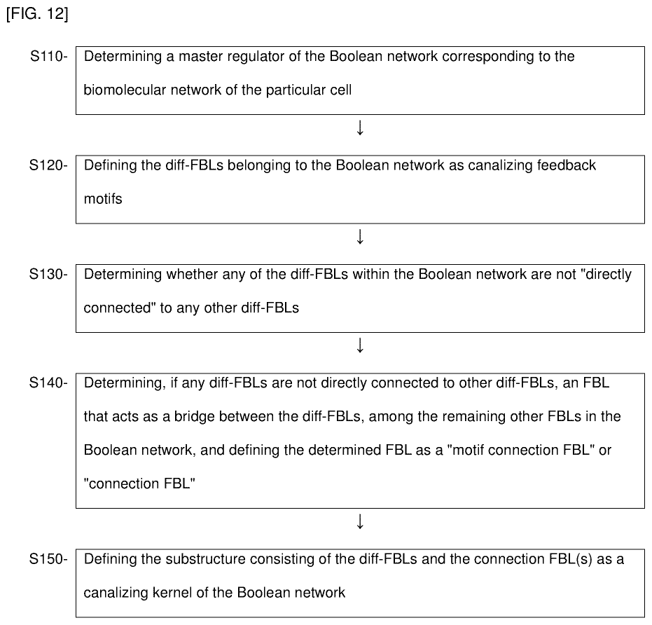

FIG. 12 is a flowchart illustrating how to define a so-called canalizing kernel, which is the core structure of a given Boolean network, according to one example of the present invention.

The method of defining the canalizing kernel may be executed after finding the master regulator illustrated in FIG. 11. Hereinafter, the method of defining the canalizing kernel will be described assuming the methodology of the present invention described in FIGS. 3 to 11.

At step (S110), the computing device may determine a master regulator of the Boolean network corresponding to the biomolecular network of the particular cell.

The master regulator may be considered to be a node corresponding to a molecule/gene/protein that determines the fate of the particular cell.

At step (S120), the computing device may define the diff-FBLs belonging to the Boolean network as canalizing feedback motifs.

At step (S130), the computing device may determine whether any of the diff-FBLs within the Boolean network are not “directly connected” to any other diff-FBLs.

If any node in one of the selected diff-FBLs is directly connected by a single link to any other node in the other selected diff-FBL, the selected diff-FBL and the other diff-FBL are “directly connected” to each other.

Referring to FIG. 5b, for example, node (x04) belonging to second diff-FBL (712) and node (x07) belonging to third diff-FBL (713) are directly connected by one link, so second diff-FBL (712) and third diff-FBL (713) are directly connected to each other. However, the first diff-FBL (711) can only be connected to the second diff-FBL (712) through node (x01) or node (x05). Further, the first diff-FBL (711) can be connected to the third diff-FBL (713) only through node (x05). Therefore, the first diff-FBL (711) is not directly connected to the second diff-FBL (712) or the third diff-FBL (713). In other words, the first diff-FBL (711) is isolated and separate from the other diff-FBLs in the Boolean network.

Thus, in the example of FIG. 5b, it is determined that all of the diff-FBLs are not directly connected in the Boolean network.

At step (S140), the computing device may determine, if any diff-FBLs are not directly connected to other diff-FBLs, an FBL that acts as a bridge between the diff-FBLs, among the remaining other FBLs in the Boolean network, and define the determined FBL as a “motif connection FBL” or “connection FBL”. There may be one or more such connection FBLs.

Based on the definition above, the other FBLs above are not diff-FBLs.

The above diff-FBLs and the above connection FBL(s) are directly connected to each other within the above Boolean network.

At step (S150), the computing device may define the substructure consisting of the diff-FBLs and the connection FBL(s) as a canalizing kernel of the Boolean network.

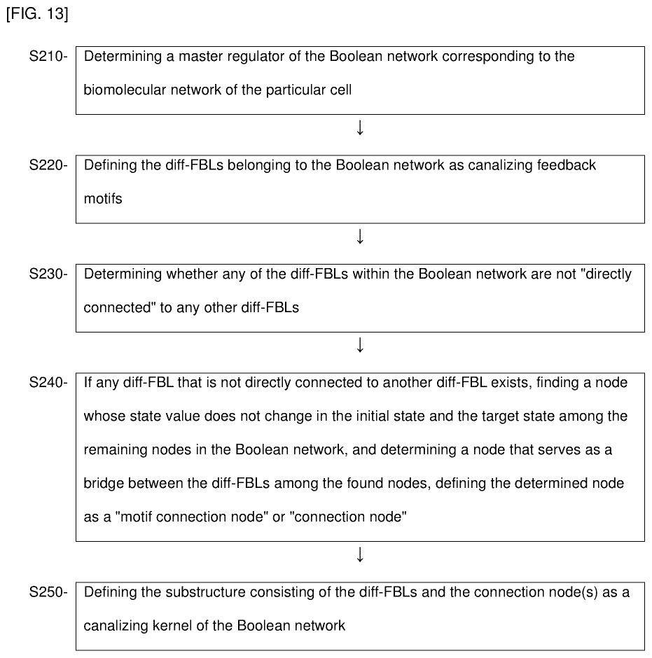

FIG. 13 is a flowchart illustrating how to define the so-called canalizing kernel, the core structure of a given Boolean network, according to another example of the present invention.

The method of defining the canalizing kernel may be executed after finding the master regulator illustrated in FIG. 11. Hereinafter, the method of defining the canalizing kernel will be described assuming the methodology of the present invention described in FIGS. 3 to 11.

Step (S210), step (S220), and step (S230) are the same as step (S110), step (S120), and step (S130) of FIG. 12, respectively.

In step (S240), the computing device may, if any diff-FBL that is not directly connected to another diff-FBL exists, find a node whose state value does not change in the initial state and the target state among the remaining nodes in the Boolean network, and determine a node that serves as a bridge between the diff-FBLs among the found nodes, defining the determined node as a “motif connection node” or “connection node”. There may be one or more such connection node.

The above diff-FBLs and the above connection node(s) are directly connected to each other within the above Boolean network.

At step (S250), the computing device may define the substructure consisting of the diff-FBLs and the connection node(s) as a canalizing kernel of the Boolean network.

Given a Boolean network modeling a state change of a particular cell, a plurality of the canalizing kernels may be defined. That is, one canalizing kernel may be determined based on a pair of initial and target states defined for the Boolean network.

For example, if the initial state is a first disease state of the particular cell and the target state is a normal state of the particular cell, the canalizing kernel may be determined to be a first canalizing kernel. In contrast, if the initial state is a second disease state of the particular cell and the target state is a normal state of the particular cell, the canalizing kernel may be determined to be a second canalizing kernel. This distinction is because the number and combination of diff-FBLs defined in the Boolean network may vary depending on the combination of the initial state and/or target state of interest with respect to the particular cell.

In this way, given one Boolean network modeling the state changes of a particular cell, the underlying core structure (canalizing kernel) that determines the fate of the particular cell may be defined differently for each combination of initial and target states of the particular cell.

Since the structure of the canalizing kernel is very small in complexity compared to the structure of the Boolean network, it has the advantage of being able to explain the core structure and operating principles (mechanisms) that are important in the entire Boolean network.

In accordance with one example of the present invention, there may be provided a non-volatile storage device on which a program readable is recorded by a computing device. The program may comprise the instructions causing the computing device to execute the steps of setting a state of each node constituting an expanded network generated from a Boolean network corresponding to a biomolecular network of a particular cell to a value of a predetermined given initial state of the Boolean network; selecting a test subject node, which is one of the nodes belonging to diff-FBLs, defined as FBLs having complementary states in the initial state of the Boolean network and in a given target state, among FBLs belonging to the Boolean network, and perturbing the selected test subject node by computer simulation; testing whether perturbing the test subject node changes the values of all nodes in the diff-FBLs from values in the initial state to values in the target state; and, if the test subject node passes the test, determining that the test subject node is a master regulator, which must be controlled to transition the Boolean network from the initial state to the target state.

In accordance with one example of the present invention, a computing device comprising the non-volatile storage and a processing part may be provided. The processing part may be configured to execute a method of identifying a cell state regulation path by reading and executing a program recorded on the non-volatile storage device.

The non-volatile storage device may be an HDD, SDD, flash memory, CD-ROM, or the like. The processing part may be an electronic circuit, including an integrated circuit such as a CPU or an AP.

### EXAMPLES FOR APPLICATION

Hereinafter, the present invention will be described in more detail with reference to the following examples. It will be apparent to those of ordinary skill in the art that these examples are provided only to more particularly explain the present invention and are not intended to limit the scope of the present invention according to the gist of the present invention.

In the examples below, the algorithm and method of the present invention were applied to a published network of cells to explore and verify a core structure (canalizing kernel) for reprogramming or trans-differentiation.

### Example 1: Exploration and Validation of Canalizing Kernel for Stem Cell Reprogramming

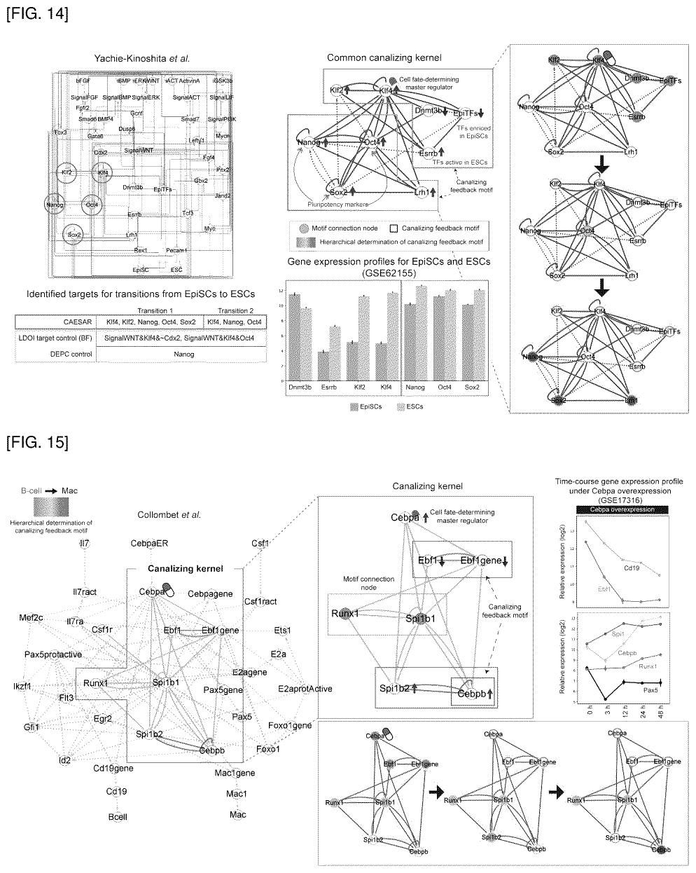

By applying the algorithm and method of the present invention to a previously known cell reprogramming network (FIG. 14, left, Yachie-kinoshita et al.), we identified a core structure (canalizing kernel) and targets that can induce a transition from epiblast-derived stem cells (EpiSCs) to embryonic stem cells (ESCs).

To verify the target derived by applying the algorithm and method of the present invention, Klf4 was overexpressed as a target and then the changes in each factor of core structure were analyzed.

As a result, as shown in FIG. 14 (right), overexpression of Klf4 led to change in the state values of Dnmt3b, Klf2, Esrrb and EpiTFs, contributing thereby to the upregulation of OSN expression levels. It suggests that our canalizing kernel would preserve of the dynamics of co-regulating genes that occur during reprogramming.

### Example 2: Exploration and Validation of Canalizing Kernel for Trans-Differentiation

To verify the effectiveness of the algorithm and method of the present invention in trans-differentiation, the algorithm and method were applied to the network involved in the B cells and macrophages specification (Collombet et al.).

Cebpa was derived as the target of the core as shown in FIG. 15 (left). As evidenced by confirming the gene expression changes of the core structure (canalizing kernel) derived in time-course patterns after Cebpa overexpression, after the Cebpa control, Ebf1 changed rapidly, and then Spi1 and Cebpb changed in that order, and the set connecting the motifs showed no change as expected (FIG. 15, right).

It means that the canalizing kernel derived by the algorithm and method of the present invention well includes the changes in the actual gene set.

According to these results, the canalizing kernel defined by the method of the present invention is very effective in deriving and designing core structure (pathway) and target (factors) in a complex network involved in determining the final cellular states.

Using the examples of the present invention described above, it will be readily apparent to those skilled in the art that various changes and modifications can be made without departing from the essential features of the invention. The subject matter of each claim may be combined with any other claim without recitation to the extent that it can be understood from this specification.

1. A method of identifying a cell state regulation path, comprising the steps,
setting, by a computing device, a state of each node constituting an expanded network generated from a Boolean network corresponding to a biomolecular network of a particular cell to a value of a predetermined given initial state of the Boolean network;
selecting, by the computing device, a test subject node, which is one of the nodes belonging to diff-FBLs, defined as FBLs having complementary states in the initial state of the Boolean network and in a given target state, among FBLs belonging to the Boolean network, and perturbs the selected test subject node by computer simulation;
testing, by the computing device, whether perturbing the test subject node changes the values of all nodes in the diff-FBLs from values in the initial state to values in the target state; and
if the test subject node passes the test, determining, by the computing device, that the test subject node is a master regulator, which must be controlled to transition the Boolean network from the initial state to the target state.

2. The method of identifying a cell state regulation path according to claim 1, further comprising the steps,
determining, by the computing device, whether any of the diff-FBLs in the Boolean network are not directly connected to other diff-FBLs;
if the diff-FBLs which are not directly connected to other diff-FBLs exist, determining, by the computing device, a set of nodes that serve as bridges connecting the diff-FBLs to each other among the remaining nodes whose state values do not change between the initial state and the target state in the Boolean network; and
defining, by the computing device, a substructure consisting of the diff-FBLs and the set of FBLs which act as bridges as a canalizing kernel of the Boolean network.

3. The method of identifying a cell state regulation path according to claim 1,
wherein the Boolean network comprises a first set of nodes corresponding to N molecules expressed in the particular cell, and a first set of links connecting the first set of nodes to each other,
wherein the first set of links includes the first type of links and the second type of links,
wherein the first type of link is a link wherein the activation of a source node connected to the starting point of the link has a positive effect on the activation of a target node connected to the destination point of the link,
wherein the second type of link is a link wherein the activation of a source node connected to the starting point of the link has a negative effect on the activation of a target node connected to the destination point of the link,
wherein the expanded network is an equivalent network to the Boolean network,
wherein the expanded network comprises a second set of nodes corresponding to the first set of nodes; a third set of nodes defined as having complementary values with respect to the second set of nodes; a fourth set of combinatorial nodes executing a logical AND operation of two nodes included in the expanded network; and a second set of links connecting the second set of nodes, the third set of nodes, and the fourth set of combinatorial nodes to each other,
wherein the second set of links consists only the first type of links.

4. The method of identifying a cell state regulation path according to claim 3,
wherein the testing step comprises
a first testing step determining a state value of the nodes of the expanded network having a determined state according to the first and second rules;
and a second testing step determining a state value of the remaining nodes of the expanded network whose state values have not been determined by the first testing step;
wherein the first rule comprises a rule for determining an active state of a second node connected to a link starting from a first node determined to be active among the nodes of the expanded network, and the second rule comprises a rule for determining an inactive state of a node defined as having a value complementary to a node determined to be active among the nodes of the expanded network,
wherein both the first node and the second node may be nodes whose state is determined according to an expression state of a single molecule of the particular cell among the nodes of the expanded network, respectively.

5. The method of identifying a cell state regulation path according to claim 4,
wherein the second testing step is to be executed only after the test subject node passes the first testing step,
wherein the test subject node is determined to have passed the first test if any of the diff-FBLs corresponding to any of the nodes in the expanded network whose state values have been determined as a result of executing the first testing step have values where the Boolean network has the target state.

6. A computing device that includes a non-volatile storage and a processing part,
wherein the processing part is configured to execute a method of identifying a cell state regulation path by reading and executing a program recorded on the non-volatile storage device,
wherein the program may comprise the instructions causing the processing part to execute the steps of setting a state of each node constituting an expanded network generated from a Boolean network corresponding to a biomolecular network of a particular cell to a value of a predetermined given initial state of the Boolean network; selecting a test subject node, which is one of the nodes belonging to diff-FBLs, defined as FBLs having complementary states in the initial state of the Boolean network and in a given target state, among FBLs belonging to the Boolean network, and perturbing the selected test subject node by computer simulation; testing whether perturbing the test subject node changes the values of all nodes in the diff-FBLs from values in the initial state to values in the target state; and, if the test subject node passes the test, determining that the test subject node is a master regulator, which must be controlled to transition the Boolean network from the initial state to the target state.

7. The computing device according to claim 6,
wherein the program may further comprise instructions for causing the processing part to further execute the steps of: determining whether any of the diff-FBLs in the Boolean network are not directly connected to other diff-FBLs; if the diff-FBLs which are not directly connected to other diff-FBLs exist, determining a set of FBLs that serve as bridges between the diff-FBLs among the remaining FBLs in the Boolean network that are not directly connected to other diff-FBLs; and defining a substructure consisting of the diff-FBLs and the set of FBLs which act as bridges as a canalizing kernel of the Boolean network.

8. The computing device according to claim 6,
wherein the Boolean network comprises a first set of nodes corresponding to N molecules expressed in the particular cell and a first set of links connecting the first set of nodes to each other,
wherein the first set of links comprises a first type of link and a second type of link,
wherein the first type of link is a link wherein activation of a source node connected to a starting point of the link has a positive effect on activation of a target node connected to a destination point of the link, and the second type of link is a link wherein activation of a source node connected to a starting point of the link has a negative effect on activation of a target node connected to a destination point of the link,
wherein the expanded network may be an equivalent network to the Boolean network,
wherein the expanded network may comprise: a second set of nodes corresponding to the first set of nodes, a third set of nodes defined as having complementary values with respect to the second set of nodes, a fourth set of combinatorial nodes executing a logical AND operation of two nodes included in the expanded network, and a second set of links connecting the second set of nodes, the third set of nodes, and the fourth set of combinatorial nodes to each other,
wherein the second set of links may comprise only the first type of links.

9. A non-volatile storage device on which a program readable by a computing device is recorded,
wherein the program may comprise the instructions causing the computing device to execute the steps of setting a state of each node constituting an expanded network generated from a Boolean network corresponding to a biomolecular network of a particular cell to a value of a predetermined given initial state of the Boolean network; selecting a test subject node, which is one of the nodes belonging to diff-FBLs, defined as FBLs having complementary states in the initial state of the Boolean network and in a given target state, among FBLs belonging to the Boolean network, and perturbing the selected test subject node by computer simulation; testing whether perturbing the test subject node changes the values of all nodes in the diff-FBLs from values in the initial state to values in the target state; and, if the test subject node passes the test, determining that the test subject node is a master regulator, which must be controlled to transition the Boolean network from the initial state to the target state.

10. The non-volatile storage device according to claim 9,
wherein the program may further comprise instructions for causing the computing device to further execute the steps of: determining whether any of the diff-FBLs in the Boolean network are not directly connected to other diff-FBLs; if the diff-FBLs which are not directly connected to other diff-FBLs exist, determining a set of nodes that serve as bridges connecting the diff-FBLs to each other among the remaining nodes whose state values do not change between the initial state and the target state in the Boolean network; and defining a substructure consisting of the diff-FBLs and the set of FBLs which act as bridges as a canal of the Boolean network.

11. The non-volatile storage device according to claim 9,
wherein the Boolean network comprises a first set of nodes corresponding to N molecules expressed in the particular cell and a first set of links connecting the first set of nodes to each other,
wherein the first set of links comprises a first type of link and a second type of link,
wherein the first type of link is a link wherein activation of a source node connected to a starting point of the link has a positive effect on activation of a target node connected to a destination point of the link, and the second type of link is a link wherein activation of a source node connected to a starting point of the link has a negative effect on activation of a target node connected to a destination point of the link,
wherein the expanded network may be an equivalent network to the Boolean network,
wherein the expanded network may comprise: a second set of nodes corresponding to the first set of nodes, a third set of nodes defined as having complementary values with respect to the second set of nodes, a fourth set of combinatorial nodes executing a logical AND operation of two nodes included in the expanded network, and a second set of links connecting the second set of nodes, the third set of nodes, and the fourth set of combinatorial nodes to each other,
wherein the second set of links may comprise only the first type of links.
